(19) United States
(12) Patent Application Publication
(10) Pub. No.: US 2016/0132656 A1
(43) Pub. Date: May 12, 2016

(54) PROCEDURE FOR THE MODELLING OF THE DIRECTION OF INCIDENT ABDOMINAL PRESSURES TOWARDS THE FEMALE PELVIC CAVITY AND THE DIRECTION OF THE REFLECTED PRESSURE IN THE PELVIC SPACE

(71) Applicant: EURL CORNIER, Paris (FR)

(72) Inventor: Edgard Cornier, Paris (FR)

(21) Appl. No.: 14/924,200

(22) Filed: Oct. 27, 2015

(30) Foreign Application Priority Data
Oct. 30, 2014 (FR) 1460474

(51) Int. Cl. G06F 19/00; A61B 5/055; A61B 5/20

(52) U.S. Cl. CPC G06F 19/3437; A61B 5/202; A61B 5/055

(57) ABSTRACT

A method for modelling the direction of incident abdominal pressures through a strait and directed towards the female pelvic cavity and for modelling the direction of pressures reflected by the pelvic paraboloid of an individual so as to allow correlating with occurrence of prolapses and incontinence in the said individual. The method includes compiling morphological data of a plane of a pelvic cavity strait by 3D cube MRI; modelling a center of gravity CG1 of the strait at a level at which the incident pressure vector penetrates in the true pelvis; modelling by 3D cube MRI a plane of a pelvic paraboloid, as well as an axis of the paraboloid; determining a CG2 reflection point of the incident vector on the plane; and determining the orientation of a reflected pressure vector.

### CROSS-REFERENCE TO RELATED APPLICATIONS

This application claims priority to French. Patent Application No. 1460474 filed Oct. 30, 2014, the entire disclosure of which is hereby explicitly incorporated by reference herein.

### BACKGROUND OF THE INVENTION

1. Field of the Invention

The present invention relates to a procedure for the modelling of the direction of incident abdominal pressures towards the female pelvic cavity and the direction of the reflected pressure in anatomical bone straits of the pelvic space.

The present invention also provides process for modelling the direction of abdominal pressures exerted in the female true pelvis, especially as a result of its standing position.

2. Description of the Related Art

The descriptions of the standard functional anatomy do not explain well the maintenance of the organs within the true pelvis of healthy women, for example the absence of muscular effort generated by pregnancies.

In a similar manner these descriptions do not properly explain the cause of prolapsus and incontinence in pathological situations.

To that extent the use of curative surgical methods using fixation processes on organs which by nature are mainly supple and mobile is not anatomically justified.

The article “Kamina”: Anatomic clinique—T4, 2012 Maloine Editeur>> (reference 1) summarises functional anatomy as it is taught today.

The model used this analysis only takes into account the solid bone structures, as they may analysed on front or profile X-ray copies.

This analysis process does not take into account the mechanical effect of the soft areas which are not seen in conventional radiology, even when the adjacent hollow organs (bladder, vagina, rectum) opacifiers are used, nor the obliqueness of the planes or the individual anatomical variations.

We thus falsely deduce that the gravity pressure and the abdominal muscular pressure are exerted along the same axis in the standing position: FIG. 14.72—page 253 (figure or FIG. 14.74—page 255 (figure II) which is summarised in FIG. 14.46—page 232 (figure III).

FIG. 1 shows the constraints and resistances of the abdominal and pelviperineal walls (median sagittal section):

A. tonic abdominal and pelviperineal walls
B. hypotonic abdominal and pelviperineal walls
F. vertical constraints
Pa. abdominal pressure
a. distension of the abdominal wall
b. perineal descent
1. resistance of the abdominal wall
2. pelviperineal muscular resistance
3. chest cavity
4. diaphragm
5. abdominal cavity
6. vertebral resistance
7. pelvic connective tissue resistance
8. anorectal body
9. perineal body

Figure II shows the orientation and position of the principal pelvic structures in upright position in the pre-vertebral zone and the infra-vertebral zone:

### A. −53 cm

### B. −89 cm

P. visceral pressure
G. line of gravity
1. pre-vertebral plane
2. upper strait
3. lower strait
4. urogenital hiatus axis
5. urogenital hiatus
6. perineal body

Figure III shows the uterine statics, the constraints and the resistances:

G. gravity pressure
P. intra-abdominal pressure
1. round lig. of uterus
2. uterosacral
3. rectum
4. anococcygeal body
5. perineal body
6. bladder
7. vagina

According to this analysis, the only modification of pressure constraints for pathological situations exerted on the perineum (cause of prolapsus) would be related to weight gain which results in an increase in gravity pressure as shown in FIG. 14.47—page 232 (figure IV).

Fig. IV shows the direction of the forces in young and elderly women:

A. abdominal parietal pressure
B. gravity pressure
P and R. constraint resultants

Furthermore, as seen in FIG. 7.2 of page 80 in reference 1 (figure V), there are different shapes of the pelvis and therefore of straits, and that in particular we can differentiate between gynecoid pelvis, platypelloid or flat pelvis, android pelvis and anthropoid pelvis.

Figure V shows the main morphological variations of the pelvis (according to the Caldweil and Moloy classification).

1. upper strait
2. pelvic arch

### SUMMARY OF THE INVENTION

The present invention provides a procedure for modelling the direction of incident abdominal pressures towards the female pelvic cavity and the direction of reflected pressures in the pelvic space of each individual so as to allow correlating with occurrence of prolapsus and incontinence in the said individual, characterised in that it includes steps that consist in compiling the morphological data of the plane of a pelvic. cavity strait by 3D cube MRI, modelling the centre of gravity CG1 of this strait at the level of which the incident pressure vector defined previously penetrates in the true pelvis, modelling by 3D cube MRI the plane of the pelvic paraboloid, as well as the axis of this paraboloid, determining the CG2 reflection point of the incident vector on this plane, and determining the orientation of the reflected pressure vector.

### BRIEF DESCRIPTION OF THE DRAWINGS

The above mentioned and other features and objects of this invention, and the manner of attaining them, will b come more apparent and the invention itself will be better understood by reference to the following description of embodiments of the invention taken in conjunction with the accompanying drawings, wherein:

FIG. 1 is a 3D cube MRI section profile section of the pelvic, cavity of a young girl;

FIG. 1A corresponds to FIG. 1, and is another 3 cube MRI section profile section of the pelvic cavity of the young girl of FIG. 1;

FIG. 2 is a 3D cube MRI section profile section of the pelvic, cavity of another young girl;

FIG. 2A corresponds to FIG. 2, and is another 3D cube MRI section profile section of the pelvic cavity of the young girl of FIG. 2;

FIG. 3 is a cube 3D MRI profile section of the pelvic cavity of an older woman;

FIG. 3A corresponds to FIG. 3, and is another cube 3D MRI profile section of the pelvic cavity of an older woman;

FIG. 4A is a view of a gynecoid pelvis;

FIG. 4B is a view of a fiat pelvis; and

FIG. 4C is a view of an android pelvis,

Corresponding reference characters indicate corresponding parts throughout the several views. The exemplifications set out herein illustrate embodiments of the disclosure and such exemplifications are not to be construed as limiting the scope of the invention in any manner.

### DETAILED DESCRIPTION

This invention proposes to model individually the direction of the incident abdominal pressure vector towards the true pelvis and the reflection plane of this incident vector.

The aggregate data are provided by 3D Cube MRI (bone tissue and soft tissue), then we describe the reflection plane of the incident vector thanks to the analysis of the data; the direction of the reflected vector can then be determined.

In fact, cube 3D MRI shows that the true pelvis is not only limited by the bone contours, but rather by a set connecting the bone contours to the adjacent soft tissues.

The straits are fixed and solid osteo-ligament zones; there is the upper strait between the pubis, the iliac bone and the sacrum as well as the posterolateral straits at the level of the sciatic foramens.

There are functional hiatuses such as he vaginal orifice or the sciatic foramen which more or less well oriented relative to the pressure axis and more or less well protected by the trophicity of the adjacent muscles.

This invention specifically proposes a mathematical model that enables the reconstitution of the shape and orientation of each physiological strait separating the pressure spaces, determining the centre of gravity of this strait at the level in which the incident pressure vector penetrates in the true pelvis, and then model the direction of the pressure vector reflected by a plane of the pelvic cavity.

To progress in this modelling, it is admitted in accordance with the invention that the sacro-coceygeal concavity is prolonged longitudinally by the perineal wall up to the genital hiatus forming a curve that can be basically comparable to a parabola.

This concavity is prolonged sideways by the sacro-sciatic ligaments and the muscles of the lateral wall of the perineum (obturator and levators) thus forming a surface that may be compared to a dome or to a paraboloid.

The pelvic cavity plane that reflects the incident pressure vector taken into consideration in accordance with the invention is the reflection plane of this paraboloid which is tangential to its edges.

The axis of reflection of this paraboloid is perpendicular to this plane.

The incident pressure vector which is reflected on this reflection plane according to an angle equal to the one made with the reflection axis, and its point of incidence corresponds to the centre of gravity of the strait.

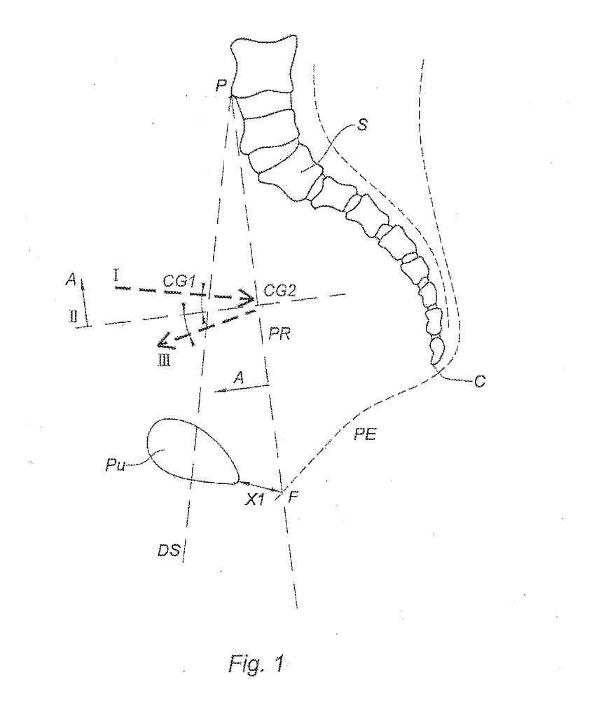

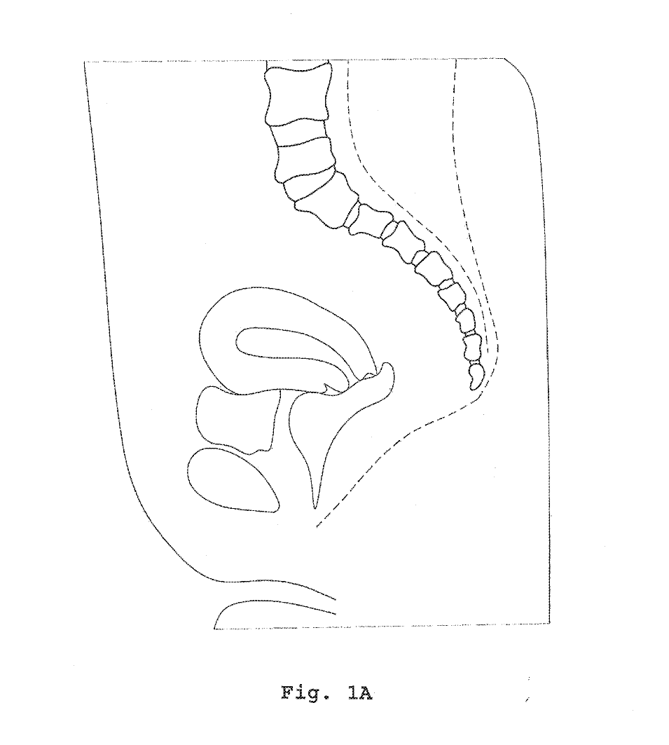

The reflected pressure vector is directed towards the front, i.e. towards the anterior axis of the pubis and the transverse umbilical muscles of the anterior wall of the abdomen (girdle) in young women (FIG. 1).

It should be noted that the pelvic paraboloid can be oriented under the elect of the abdomino-pelvic muscular contraction with modifications related to the age and parity, i.e. the number of pregnancies and births.

In fact, as seen in FIG. 1 which corresponds to a 3D cube MRI section profile section (FIG. 1A) of the pelvic cavity of a young girl, the distance X₁ between the lower edge of the pubis and the perineal corner is of the order of 2.5 cm.

Under the effect of the simultaneous muscular contraction of the inferior abdominal wall and of the perineum, this distance is reduced by about 5 mm and the axis of the paraboloid is oriented towards the top as represented by the A arrows.

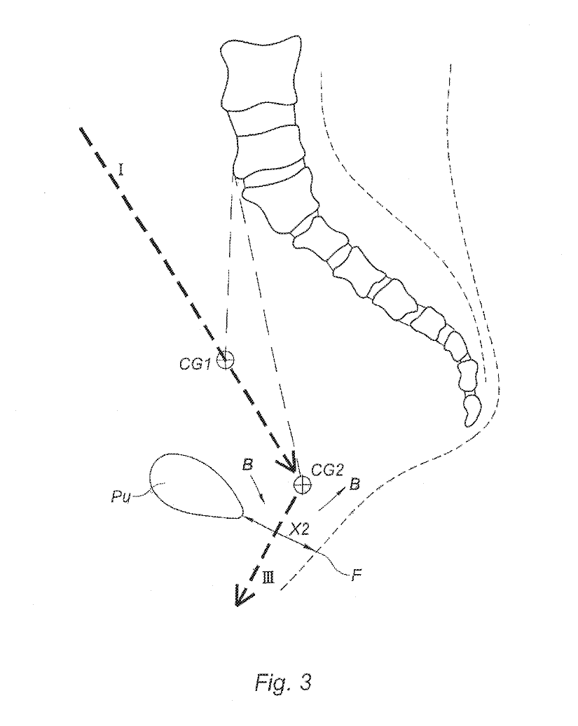

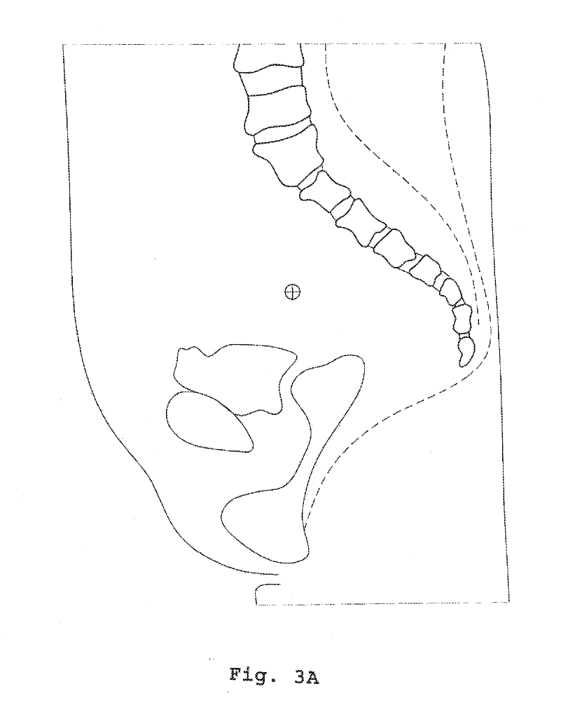

Conversely, according to FIG. 3 which corresponds to a cube 3D MRI profile section (FIG. 3A) of the pelvic cavity of an older woman and who had several children, the distance X₂ between the lower edge of the pubis and the perineal corner is higher and the paraboloid axis is oriented notably toward the bottom as represented by the B arrows.

As a result the fleeted pressure vector is directed even lower towards the vaginal hiatus.

The invention thus allows modelling a strait, its centre of gravity and the incident pressure vector directed towards the true pelvis from this strait using 3D cube MRI data acquisition.

It then allows the reconstitution of the pelvic paraboloid plane from the data acquired, and therefore to describe the orientation of the pressure vector reflected by the existing soft structures.

In each case, the mathematical model allows determining the strait and the associated pelvic paraboloid, its centre of gravity and the direction of the reflected pressure vector.

We can thus determine in each case if the reflected pressure vector is directed towards the top, preventing the occurrence of prolapsus or towards the bottom, in the direction of the genital hiatus for example.

This modelling can also be applied to the acquisition of other hiatuses, such as the posterolateral or obturator sciatic spaces.

Therefore, it is possible to analyse the incidence and the reflection of the abdominal pressure vector towards the pudendal zones when the pelvic muscles have been altered.

Each case may be thus analysed as a function of all these anatomical specificities.

It should therefore be rioted that there are numerous anatomical differences between the shapes of the bony pelvis in women.

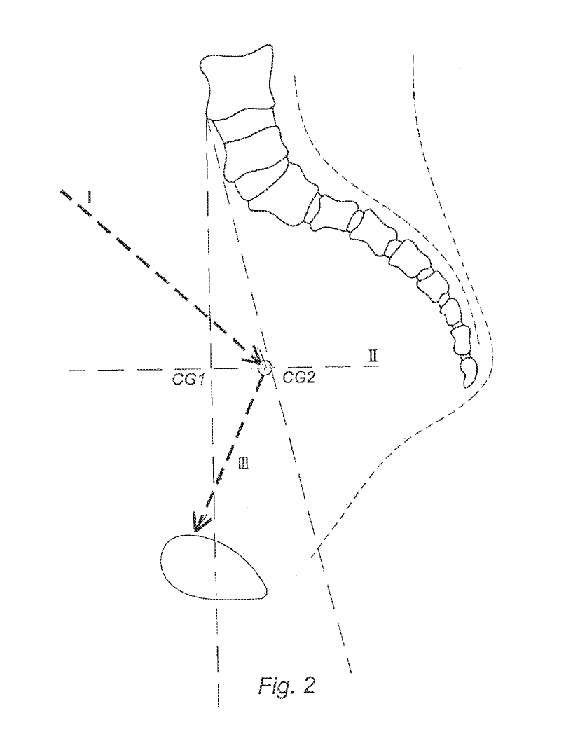

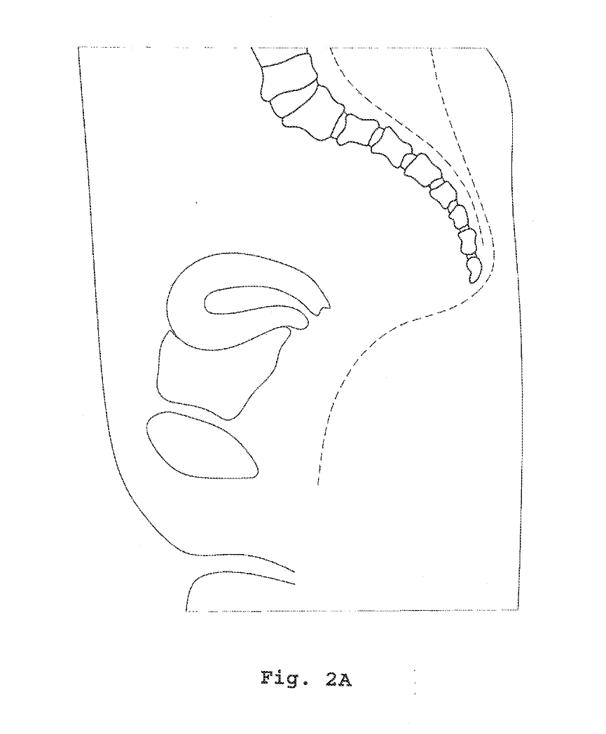

For example, as seen in FIG. 2 which corresponds to a 3D cube MRI profile section (FIG. 2A) of the pelvic cavity of a young woman with a different morphology from the previous example, there are flatter sacra more or less prolonged by the coccyx and the fibromuscular perineum.

The reflected vector is directed towards the top and towards the front unlike that of FIG. 3 (woman with a prolapsus).

The modelling in accordance with the invention allows defining the centre of gravity of the pressure vector for a strait, irrespective of the shape of the pelvis.

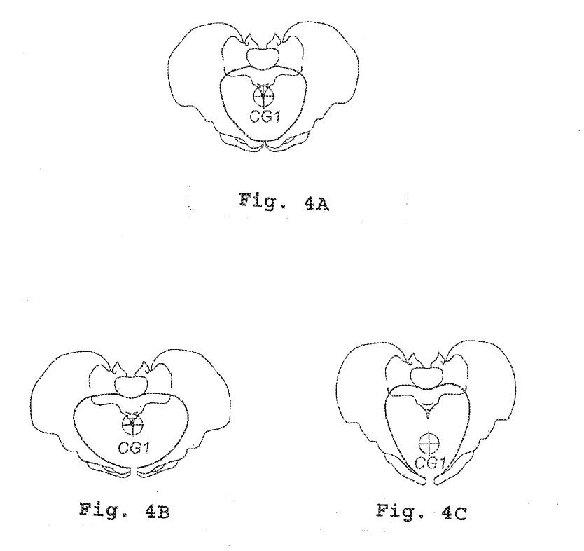

According to FIGS. 4A, 4B and 4C this centre of gravity CG1 of the strait represented in figure V is positioned differently depending on the shape of the pelvis and is shifted towards the back in the case of a gynecoid pelvis (FIG. 4A), it is located in a central position in the case of a flat pelvis (FIG. 4B) and is shifted towards the front in the case of an android pelvis (FIG. 4C).

According to the invention, the modelling of the data compiled by the 3D cube MRI thus allows modelling the reflected pressure vector as a function of the anatomical variations of each individual.

Therefore, the subject of this invention is a procedure for the modelling of the direction of incident abdominal pressures towards the female pelvic cavity and the direction of the reflected pressure in anatomical bone straits of the pelvic space of a person in order to understand the mechanisms of a prolapsus or pudendal neuralgia in this person.

It also allows following the effects of the correction of these disorders.

This process comprises steps that consist in:
compiling the morphological data of the plane of a pelvic cavity strait by D cube MRI,
modelling the centre of gravity CG1 of this strait at the level of which the incident pressure vector penetrates in the true pelvis,
modelling by 3D cube MRI the plane of the pelvic paraboloid, as well as the axis of this paraboloid,
determining the CG2 reflection point of the incident vector on this plane, and
determining the orientation of the reflected pressure vector.

In accordance with another characteristic of the invention, we refine the determination of the centre of gravity CG1 of the strait taking into account the variations in the density of the intestinal content, in order to define a weighted centre of gravity CG1′ of the strait from which we determine the orientation of the reflected pressure vector and we thus deduce the prolapsus and incontinence risks.

The data acquired by 3D cube MRI also allow differentiating between the solid materials of the intestinal content and the soft matters that comply with viscous body mechanics.

In particular, the invention allows modelling by 3D cube MRI the upper bony strait as defined by the anatomy and its centre of gravity CG1.

3D cube MRI also shows the pelvic paraboloid and its reflection plane.

The modelling allows defining the orientation of the pressure vector for each individual.

As an example, FIG. 1 represents the 3D cube MRI profile section of a gynecoid superior strait of a young girl represented by the line DS which connects the promontory P of the sacrum S to the upper third of the pubis PU.

The centre of gravity CG1 of this upper strait is defined by modelling.

The pelvic paraboloid is represented by the sacrum S and the coccyx C which are prolonged by the perineal wall PE to the genital hiatus forming a curve similar to a parabola.

3D cube MRI allows determining the reflection plane PR of this paraboloid which is delimited by the promontory P of the sacrum S and the perineal fourchette F i.e. the fibrous corner of the perineum.

3D cube MRI also allows determining the axis II of the paraboloid.

The incident pressure vector 1 passes through a line connecting the navel to the centre of gravity CG1 of the upper strait and continues towards the reflection plane of the pelvic paraboloid where it is reflected according to axis II of the paraboloid at the reflection point CG2.

The reflected pressure vector is oriented towards the bottom but more or less high to pass either towards the pubis and the abdominal girdle, or lower below towards the genital hiatus.

The modelling allows defining it.

The invention thus allows modelling the data acquired by 3D cube MRI for each case to reconstitute the surface of a strait and deduce the centre of gravity and the direction of the incident pressure vector and then the pressure vector reflected by the plane of a parabaloid reflecting the vectors.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1-2. (canceled)

3. A method for modelling the direction of incident abdominal pressures towards a female pelvic cavity and a direction of reflected pressures in a pelvic space of an individual for use in correlating with an occurrence of prolapses and incontinence in the individual, said method comprising the following steps:
compiling morphological data of a plane of a pelvic cavity strait by 3D cube MRI;
modelling a center of gravity CG1 of the strait at a level at which an incident pressure vector penetrates in the true pelvis,
modelling by 3D cube MRI a plane of a pelvic paraboloid and an axis of the paraboloid;
determining a CG2 reflection point of an incident vector on the plane; and
determining the orientation of a reflected pressure vector.

4. The method of claim 3, comprising the additional step, prior to said step of determining the orientation of a reflected pressure vector, of refining the determination of the center of gravity CG1 of the upper strait using variations in a density of intestinal content to define a weighted center of gravity (CG1′).
